(19) United States
(12) Patent Application Publication
(10) Pub. No.: US 2026/0294980 A1
(43) Pub. Date: Oct. 1, 2026

(54) LIQUID FOR PRESERVATION OR TRANSPLANTATION OF ENCAPSULATED ISLET

(71) Applicant: OTSUKA PHARMACEUTICAL FACTORY, INC., Naruto-shi, Tokushima (JP)

(72) Inventors: Yu HAYAMI, Naruto-shi, Tokushima (JP); Naho IIZUKA, Naruto-shi, Tokushima (JP); Yamato OKITA, Naruto-shi, Tokushima (JP)

(21) Appl. No.: 18/992,809

(22) PCT Filed: Jul. 11, 2023
(86) PCT No.: PCT/JP2023/025519

§ 371 Date: Jan. 9, 2025

(30) Foreign Application Priority Data
Jul. 12, 2022 (JP) 2022-111897

(51) Int. Cl. A61K 35/39; A61K 47/02; A61K 47/12; A61K 47/26

(52) U.S. Cl. CPC A61K 35/39; A61K 47/02; A61K 47/12; A61K 47/26

(57) ABSTRACT

To provide a means of more efficiently performing encapsulated islet transplantation. A liquid for preservation or transplantation of an encapsulated islet, including trehalose or a derivative thereof or a salt thereof.

### TECHNICAL FIELD

Techniques related to a liquid for preservation or transplantation of an encapsulated islet and the use thereof are disclosed.

### BACKGROUND ART

Diabetes is a disease that occurs when the function of the pancreas for controlling blood sugar is impaired, and there are mainly two types of diabetes, type I diabetes and type II diabetes. Type I diabetes develops at relatively young age and causes symptoms in which cells that produce insulin are strongly affected by an autoimmune reaction, whereas type II diabetes develops in middle age and is caused by poor use of insulin. In Japan, 5% of the diabetic patients correspond to type I diabetes, and it is estimated that there are about 300,000 diabetic patients. In many cases, these diabetic patients control blood glucose by insulin therapy, but are not always immune from developing complications such as nephropathy, retinopathy, and neuropathy. In addition, there are many cases where blood glucose cannot be controlled even by insulin therapy.

An alternative treatment of diabetes instead of insulin therapy includes islet transplantation. Islets (also called islets of Langerhans) are a group of endocrine cells that are scattered in the form of islands in the tissues of the pancreas and contain R cells that secrete insulin. Islet transplantation is a transplantation therapy in which islets are separated from a pancreas provided from a donor, a dispersion of the islets is prepared, and then the dispersion is percutaneously administered to a patient.

Islet transplantation has the following advantages. (1) Percutaneous administration is possible, and thus laparotomy or general anesthesia is not required, and the burden on the patient during transplantation surgery is small. (2) The dose of the immunosuppressive agent after transplantation is smaller than that in the case of the transplantation of the pulp. (3) The pancreas of an organ donor who is not applied in pancreatic transplantation, such as an organ donor who died from cardiac arrest or arteriosclerosis, can also be applied. In addition, as a means for further reducing the dose of an immunosuppressive agent after transplantation, encapsulated islets enclosing the islets in minute microcapsules are known (PTL 1 and PTL 2).

Many of the β cells constituting the islets are deactivated over time after the separation and purification of the islets, and thus it is desirable that the prepared islets is appropriately preserved and transplanted in a short time so as not to lose vitality thereof as much as possible. However, it takes a certain time (for example, about 10 to 18 hours) from the surgery of extracting the pancreas from the donor to the preparation of islets for transplantation. In addition, even when preparation for islet transplantation is ready, transplantation surgery may not be performed immediately due to poor patient's physical conditions or the like.

### CITATION LIST

### Patent Literatures

PTL 1: WO2021/256451
PTL 2: WO2021/153365

### SUMMARY OF INVENTION

### Technical Problem

An object is to provide a means to more efficiently perform encapsulated islet transplantation.

### Solution to Problem

In order to solve the above problems, intensive studies have been performed, and it has been found that it is possible to use a solution common to preservation and transplantation of encapsulated islets. Such findings are further studied, and the invention represented by the following is provided.

### Clause 1

A liquid for preservation or transplantation of an encapsulated islet, including trehalose or a derivative thereof or a salt thereof.

### Clause 2

The liquid according to clause 1, being an isotonic liquid.

### Clause 3

The liquid according to claim 2, in which the isotonic liquid is Ringer's lactate liquid.

### Clause 4

The liquid according to any one of clauses 1 to 3, in which a concentration of trehalose or a derivative thereof or a salt thereof is 2.0 to 6.0 (w/v) %.

### Clause 5

The liquid according to any one of clauses 1 to 4, being used for preventing tissue adhesion of the encapsulated islet.

### Clause 6

A method for preservation of an encapsulated islet, including a step of preserving the encapsulated islet in the liquid according to any of clauses 1 to 5.

### Clause 7

The liquid according to any of clauses 1 to 5, further including an encapsulated islet.

### Clause 8

A method for treating diabetes, including administering the liquid according to clause 7 to a subject in need of islet transplantation.

### Advantageous Effects of Invention

When the encapsulated islet is transplanted, the transplantation can be performed without transferring the preserved encapsulated islet to a transplantation liquid different from a preservation liquid. This allows encapsulated islet transplantation to be more efficient (alternatively, allows reducing the time required for encapsulated islet transplantation). In addition, there is provided excellent function of suppressing adhesion of the encapsulated islet to tissues after transplantation.

### BRIEF DESCRIPTION OF DRAWINGS

FIG. 1 shows changes in body weight (average value) measured for a non-preserved group, a control group, and a test group.

FIG. 2 shows changes in blood glucose levels (average values) at the time of satiation measured for the non-preserved group, the control group, and the test group.

FIG. 3 shows DTZ-stained photographs of the encapsulated islet collected from the non-preserved group, the control group, and the test group.

FIG. 4 shows the insulin secretion amount and the glucose responsiveness measured for the encapsulated islet collected from the non-preserved group, the control group, and the test group.

### DESCRIPTION OF EMBODIMENTS

### 1. Liquid for Preservation or Transplantation

The liquid for preservation or transplantation of the encapsulated islet preferably contains trehalose or a derivative thereof or a salt thereof (these may be collectively referred to as “trehalose”). Examples of the trehalose include α,α-trehalose that is a disaccharide in which two α-glucoses are 1,1-glycosidically bonded, α,β-trehalose that is a disaccharide in which α-glucose and β-glucose are 1,1-glycosidically bonded, and β,β-trehalose that is a disaccharide in which two β-glucoses are 1,1-glycosidically bonded. Of these, α,α-trehalose is preferable. These trehalose can be produced by any known method such as chemical synthesis, production by microorganisms, and production by enzymes, but commercially available products can also be used. Examples thereof include commercially available products such as α,α-trehalose (manufactured by Hayashibara Co., Ltd., or manufactured by FUJIFILM Wako Pure Chemical Corporation).

The derivative of trehalose is not particularly limited, and examples thereof include glycosyl trehalose in which one or more glucose units are bonded to trehalose of a disaccharide. Glycosyl trehaloses include glucosyl trehalose, maltosyl trehalose, maltotriosyl trehalose, and the like.

The salt of trehalose or a derivative thereof is not particularly limited as long as it is pharmaceutically acceptable. Examples thereof include acid addition salts such as hydrochloride, hydrobromide, hydroiodide, phosphate, nitrate, sulfate, acetate, propionate, toluenesulfonate, succinate, oxalate, lactate, tartrate, glycolate, methanesulfonate, butyrate, valerate, citrate, fumarate, maleate, and malate; metal salts such as sodium salt, potassium salt, and calcium salt; ammonium salts; and alkylammonium salts. It is preferable that these salts have the same effect as in the case of trehalose when used as a solution. These salts may form hydrates or solvates. In addition, two or more types can be appropriately combined and used. The liquid for preservation or transplantation may contain only one type of trehalose or may contain two or more types of trehalose in combination.

The concentration of trehalose in the liquid for preservation or transplantation is not particularly limited, and for example, the lower limit is 0.1 (w/v) % or more, preferably 0.3 (w/v) or more, more preferably 0.6 (w/v) % or more, still more preferably 1.0 (w/v) or more, and most preferably 2.0 (w/v) t or more in terms of trehalose. In addition, the upper limit is, for example, 40 (w/v) % or less, preferably 20 (w/v) % or less, more preferably 15 (w/v) % or less, still more preferably 10% (w/v) % or less, and most preferably 6.0 (w/v) % or less in terms of trehalose. Any of these lower and upper limit values can be combined. For example, the concentration of trehalose in the liquid for preservation or transplantation is within a range of 0.1 to 40 (w/v) %, preferably 0.3 to 20 (w/v) %, more preferably 0.6 to 15 (w/v) %, still more preferably 1.0 to 10% (w/v) %, and most preferably 2.0 to 6.0 (w/v) % in terms of trehalose.

The liquid for preservation or transplantation may be any liquid capable of transplanting and preserving the encapsulated islet (for example, isotonic liquid, hypotonic liquid, or hypertonic liquid), and is preferably an isotonic liquid. Herein, the “isotonic liquid” means a solution having an osmotic pressure substantially equal to the osmotic pressure of a body liquid or a cell liquid, and specifically means a solution having an osmotic pressure within a range of 250 to 380 mOsm/L. The “hypotonic liquid” means a liquid having an osmotic pressure lower than the osmotic pressure of a body liquid or a cell liquid, and specifically means a liquid having an osmotic pressure of less than 250 mOsm/L. The hypotonic liquid is preferably a hypotonic solution (specifically, a liquid having an osmotic pressure within a range of less than 100 to 250 mOsm/L) that does not rupture cells. The “hypertonic liquid” means a liquid having an osmotic pressure higher than the osmotic pressure of a body liquid or a cell liquid, and specifically means a liquid having an osmotic pressure of more than 380 mOsm/L (preferably in a range of more than 380 mOsm/L to 1000 mOsm/L).

The isotonic liquid is not particularly limited as long as it is an isotonic liquid in which the salt concentration, the glucose concentration, and the like are adjusted by sodium ions, potassium ions, calcium ions, and/or the like so as to have almost the sane osmotic pressure as the body liquid or the cell liquid. Specific examples thereof include physiological saline, physiological saline having a buffering effect (for example, PBS, Tris Buffered Saline: TBS], HEPES buffered saline), Ringer's liquid, Ringer's lactate liquid, Ringer's acetate liquid, Ringer's bicarbonate liquid, 5% glucose aqueous liquid, basal medium for culturing animal cells (for example, DMEM, EMEM, RPMI-1640, α-MEM, F-12, F-10, and M-199), and a liquid prepared with an isotonic agent (for example, glucose, D-sorbitol, D-mannitol, lactose, and sodium chloride). Of these, Ringer's lactate liquid is preferable. The isotonic liquid may be a commercially available liquid or a liquid prepared by itself. Examples of commercially available products include OTSUKA NORMAL SALINE (manufactured by Otsuka Pharmaceutical Factory, Inc.) (physiological saline liquid), Ringer's liquid “OTSUKA” (manufactured by Otsuka Pharmaceutical Factory, Inc.) (Ringer's lactate liquid), Lactec (registered trademark) Injection (manufactured by Otsuka Pharmaceutical Factory, Inc.) (lactated Ringer's liquid), Veen (registered trademark)-F Injection (manufactured by Fuso Pharmaceutical Industries, Ltd.) (acetic acid Ringer's liquid), OTSUKA GLUCOSE INJECTION 5% (manufactured by Otsuka Pharmaceutical Factory, Inc.) (51 glucose aqueous liquid), and BICANATE (registered trademark) Injection (manufactured by Otsuka Pharmaceutical Factory, Inc.) (bicarbonated Ringer's liquid).

The pH of the liquid for preservation or transplantation is not particularly limited as long as it is suitable for transplantation and preservation of the encapsulated islet, and may be, for example, within the range of 6.5 to 8.5. For example, the pH may be 6.5 to 8.4; 6.5 to 8.3; 6.5 to 8.2; 6.5 to 8.1; 6.5 to 8.0; 6.5 to 7.9; 6.5 to 7.8; 6.5 to 7.7; 6.5 to 7.6; 6.5 to 7.5; 6.5 to 7.4; 6.5 to 7.3; 6.5 to 7.2; 6.5 to 7.1; 6.5 to 7.0; 6.5 to 6.9; 6.5 to 6.8; 6.6 to 8.5; 6.7 to 8.5; 6.8 to 8.5; 6.9 to 8.5; 7.0 to 8.5; 7.1 to 8.5; 7.2 to 8.5; 7.3 to 8.5; 7.4 to 8.5; 7.5 to 8.5; 7.6 to 8.5; 7.7 to 8.5; 7.8 to 8.5; 7.9 to 8.5; 8.0 to 8.5; 8.1 to 8.5; 8.2 to 8.5; 6.6 to 8.4; 6.6 to 8.3; 6.6 to 8.2; 6.6 to 8.1; 6.6 to 8.0; 6.6 to 7.9; 6.6 to 7.8; 6.6 to 7.7; 6.6 to 7.6; 6.6 to 7.5; 6.6 to 7.4; 6.6 to 7.3; 6.6 to 7.2; 6.6 to 7.1; 6.6 to 7.0; 6.6 to 6.9; 6.7 to 8.4; 6.7 to 8.3; 6.7 to 8.2; 6.7 to 8.1; 6.7 to 8.0; 6.7 to 7.9; 6.7 to 7.8; 6.7 to 7.7; 6.7 to 7.6; 6.7 to 7.5; 6.7 to 7.4; 6.7 to 7.3; 6.7 to 7.2; 6.7 to 7.1; 6.7 to 7.0; 6.8 to 8.4; 6.8 to 8.3; 6.8 to 8.2; 6.8 to 8.1; 6.8 to 8.0; 6.8 to 7.9; 6.8 to 7.8; 6.8 to 7.7; 6.8 to 7.6; 6.8 to 7.5; 6.8 to 7.4; 6.8 to 7.3; 6.8 to 7.2; 6.8 to 7.1; 6.9 to 8.4; 6.9 to 8.3; 6.9 to 8.2; 6.9 to 8.1; 6.9 to 8.0; 6.9 to 7.9; 6.9 to 7.8; 6.9 to 7.7; 6.9 to 7.6; 6.9 to 7.5; 6.9 to 7.4; 6.9 to 7.3; 6.9 to 7.2; 7.0 to 8.4; 7.0 to 8.3; 7.0 to 8.2; 7.0 to 8.1; 7.0 to 8.0; 7.0 to 7.9; 7.0 to 7.8; 7.0 to 7.7; 7.0 to 7.6; 7.0 to 7.5; 7.0 to 7.4; 7.0 to 7.3; 7.1 to 8.4; 7.1 to 8.3; 7.1 to 8.2; 7.1 to 8.1; 7.1 to 8.0; 7.1 to 7.9; 7.1 to 7.8; 7.1 to 7.7; 7.1 to 7.6; 7.1 to 7.5; 7.1 to 7.4; 7.2 to 8.4; 7.2 to 8.3; 7.2 to 8.2; 7.2 to 8.1; 7.2 to 8.0; 7.2 to 7.9; 7.2 to 7.8; 7.2 to 7.7; 7.2 to 7.6; 7.2 to 7.5; 7.3 to 8.4; 7.3 to 8.3; 7.3 to 8.2; 7.3 to 8.1; 7.3 to 8.0; 7.3 to 7.9; 7.3 to 7.8; 7.3 to 7.7; 7.3 to 7.6; 7.4 to 8.4; 7.4 to 8.3; 7.4 to 8.2; 7.4 to 8.1; 7.4 to 8.0; 7.4 to 7.9; 7.4 to 7.8; 7.4 to 7.7; 7.5 to 8.4; 7.5 to 8.3; 7.5 to 8.2; 7.5 to 8.1; 7.5 to 8.0; 7.5 to 7.9; 7.5 to 7.8; 7.6 to 8.4; 7.6 to 8.3; 7.6 to 8.2; 7.6 to 8.1; 7.6 to 8.0; 7.6 to 7.9; 7.7 to 8.4; 7.7 to 8.3; 7.7 to 8.2; 7.7 to 8.1; 7.7 to 8.0; 7.8 to 8.4; 7.8 to 8.3; 7.8 to 8.2; 7.8 to 8.1; 7.9 to 8.4; 7.9 to 8.3; 7.9 to 8.2; 8.0 to 8.4; 8.0 to 8.3; 8.1 to 8.3; and the like.

In one embodiment, the liquid for preservation or transplantation preferably does not contain a substance unsuitable for mammalian cell transplantation. Examples of substances that are unsuitable for mammalian cell transplantation include biologically derived components (for example, serum or serum-derived components (for example, albumin)), dimethyl sulfoxide [ENSO], glycerin, ethylene glycol, trimethylene glycol, dimethylacetamide, polyethylene glycol [PEG], and polyvinyl pyrrolidone.

The type of the islet used for the encapsulated islet to be transplanted (and preserved) using the liquid for preservation or transplantation is not particularly limited. The islet preferably includes insulin-producing R cells, glucagon-containing a cells, somatostatin secreting delta cells, and pancreatic polypeptide containing cells (PP cells). The islet is preferably mostly insulin-producing P cells. The origin of the islet can be selected according to the purpose, and is preferably human, pig, mouse, rat, monkey, or dog. In one embodiment, the islet is preferably derived from a pig and preferably a neonatal pig (for example, 3 days to 4 weeks after birth or 7 days to 3 weeks after birth). The islet can be obtained by any method known in the art.

The size of the islet is preferably 50 μm or more and 400 μm or less, and more preferably 50 μm or more and 350 μm or less. The size of the islet can be measured using a micrometer of a microscope. The islet preferably contains 10% or more of β cells as a characteristic. The upper limit of the proportion of β cells is not particularly limited, but is, for example, 80%.

In one embodiment, the encapsulated islet is preferably an encapsulated islet as described in PTL 1 or PTL 2. In one embodiment, it is preferable that the encapsulated islet has a structure in which a core is covered with triple membranes, in which the core contains the islet, the first and third membranes from the inside of the triple membranes contain alginic acid, and the second membrane from the inside contains polyornithine. In one embodiment, the encapsulated islet may be covered with more than triple membranes (for example, quadruple membranes, quintuple membranes, sextuple membranes, septuple membranes, or octuple membranes).

The fact that the membrane contains alginic acid means that the membrane mainly includes alginic acid. The fact that the membrane contains polyornithine means that the membrane mainly includes polyornithine. The encapsulated islet preferably contains one to five islets in one capsule.

The encapsulated islet preferably has an average diameter of 400 μm or more, more preferably 420 μm or more, from the viewpoint of suppressing fibrosis. The upper limit of the average diameter is not particularly limited, but can be set to, for example, 700 μm or less, 600 μm or less, or 500 μm or less. The average diameter can be measured by a measurement method adopted in Examples described later.

The encapsulated islet can be prepared by the methods described in PTL 1 and PTL 2 and the like.

The above-described liquid for preservation or transplantation can be used for transplantation as it is without transferring the preserved encapsulated islet to another liquid for transplantation. Thus, in one embodiment, the liquid for preservation or liquid for transplantation can be not only a liquid for preservation but also liquid for transplantation for the islet. In addition, the liquid for preservation or transplantation is excellent in the function of suppressing adhesion of the encapsulated islet after transplantation to tissues (for example, liver). Therefore, the liquid for preservation or transplantation can be used for preventing adhesion of the encapsulated islet to tissues.

The preservation period and the preservation temperature during preserving the encapsulated islet using the liquid for preservation or transplantation of the present invention are as described in “2. Preservation method” described later.

### 2. Preservation Method

There is provided a method for preserving the encapsulated islet, including a step of preserving the encapsulated islet in the liquid for preservation or liquid for transplantation as described above.

The preservation period is arbitrary and is not particularly limited. In one embodiment, the preservation period may be several hours to several days. The several hours are, for example, 0 hours, 2 hours, 4 hours, 6 hours, 8 hours, 10 hours, 12 hours, 14 hours, 16 hours, 18 hours, 20 hours, or 22 hours. The several days are, for example, 1 day, 2 days, 3 days, 4 days, 5 days, 6 days, 7 days, 8 days, 9 days, 10 days, 11 days, or 12 days. Examples of the preservation period include 0 hours to 12 days; 0 hours to 11 days; 0 hours to 10 days; 0 hours to 9 days; 0 hours to 8 days; 0 hours to 7 days; 0 hours to 6 days; 0 hours to 5 days; 0 hours to 4 days; 0 hours to 3 days; 0 hours to 2 days; 0 hours to 1 day; 2 hours to 12 days; 2 hours to 11 days; 2 hours to 10 days; 2 hours to 9 days; 2 hours to 8 days; 2 hours to 7 days; 2 hours to 6 days; 2 hours to 5 days; 2 hours to 4 days; 2 hours to 3 days; 2 hours to 2 days; 2 hours to 1 day; 4 hours to 12 days; 4 hours to 11 days; 4 hours to 10 days; 4 hours to 9 days; 4 hours to 8 days; 4 hours to 7 days; 4 hours to 6 days; 4 hours to 5 days; 4 hours to 4 days; 4 hours to 3 days; 4 hours to 2 days; 4 hours to 1 day; 6 hours to 12 days; 6 hours to 11 days; 6 hours to 10 days; 6 hours to 9 days; 6 hours to 8 days; 6 hours to 7 days; 6 hours to 6 days; 6 hours to 5 days; 6 hours to 4 days; 6 hours to 3 days; 6 hours to 2 days; 6 hours to 1 day; 8 hours to 12 days; 8 hours to 11 days; 8 hours to 10 days; 8 hours to 9 days; 8 hours to 8 days; 8 hours to 7 days; 8 hours to 6 days; 8 hours to 5 days; 8 hours to 4 days; 8 hours to 3 days; 8 hours to 2 days; 8 hours to 1 day; 10 hours to 12 days; 10 hours to 11 days; 10 hours to 10 days; 10 hours to 9 days; 10 hours to 8 days; 10 hours to 7 days; 10 hours to 6 days; 10 hours to 5 days; 10 hours to 4 days; 10 hours to 3 days; 10 hours to 2 days; 10 hours to 1 day; 12 hours to 12 days; 12 hours to 11 days; 12 hours to 10 days; 12 hours to 9 days; 12 hours to 8 days; 12 hours to 7 days; 12 hours to 6 days; 12 hours to 5 days; 12 hours to 4 days; 12 hours to 3 days; 12 hours to 2 days; 12 hours to 1 day; 14 hours to 12 days; 14 hours to 11 days; 14 hours to 10 days; 14 hours to 9 days; 14 hours to 8 days; 14 hours to 7 days; 14 hours to 6 days; 14 hours to 5 days; 14 hours to 4 days; 14 hours to 3 days; 14 hours to 2 days; 14 hours to 1 day; 16 hours to 12 days; 16 hours to 11 days; 16 hours to 10 days; 16 hours to 9 days; 16 hours to 8 days; 16 hours to 7 days; 16 hours to 6 days; 16 hours to 5 days; 16 hours to 4 days; 16 hours to 3 days; 16 hours to 2 days; 16 hours to 1 day; 18 hours to 12 days; 18 hours to 11 days; 18 hours to 10 days; 18 hours to 9 days; 18 hours to 8 days; 18 hours to 7 days; 18 hours to 6 days; 18 hours to 5 days; 18 hours to 4 days; 18 hours to 3 days; 18 hours to 2 days; 18 hours to 1 day; 20 hours to 12 days; 20 hours to 11 days; 20 hours to 10 days; 20 hours to 9 days; 20 hours to 8 days; 20 hours to 7 days; 20 hours to 6 days; 20 hours to 5 days; 20 hours to 4 days; 20 hours to 3 days; 20 hours to 2 days; 20 hours to 1 day; 22 hours to 12 days; 22 hours to 11 days; 22 hours to 10 days; 22 hours to 9 days; 22 hours to 8 days; 22 hours to 7 days; 22 hours to 6 days; 22 hours to 5 days; 22 hours to 4 days; 22 hours to 3 days; 22 hours to 2 days; and 22 hours to 1 day.

The preservation temperature is arbitrary and is not particularly limited. In one embodiment, the preservation temperature may be 0 to 40° C. Examples of the preservation temperature include 0 to 40° C.; 0 to 38° C.; 0 to 36° C.; 0 to 34° C.; 0 to 32° C.; 0 to 30° C.; 0 to 28° C.; 0 to 26° C.; 0 to 24° C.; 0 to 22° C.; 0 to 20° C.; 0 to 18° C.; 0 to 16° C.; 0 to 14° C.; 0 to 12° C.; 0 to 10° C.; 0 to 8° C.; 0 to 6° C.; 0 to 4° C.; 0 to 2° C.; 2 to 40° C.; 2 to 38° C.; 2 to 36° C.; 2 to 34° C.; 2 to 32° C.; 2 to 30° C.; 2 to 28° C.; 2 to 26° C.; 2 to 24° C.; 2 to 22° C.; 2 to 20° C.; 2 to 18° C.; 2 to 16° C.; 2 to 14° C.; 2 to 12° C.; 2 to 10° C.; 2 to 8° C.; 2 to 6° C.; 2 to 4° C.; 4 to 40° C.; 4 to 38° C.; 4 to 36° C.; 4 to 34° C.; 4 to 32° C.; 4 to 30° C.; 4 to 28° C.; 4 to 26° C.; 4 to 24° C.; 4 to 22° C.; 4 to 20° C.; 4 to 18° C.; 4 to 16° C.; 4 to 14° C.; 4 to 12° C.; 4 to 10° C.; 4 to 8° C.; 4 to 6° C.; 6 to 40° C.; 6 to 38° C.; 6 to 36° C.; 6 to 34° C.; 6 to 32° C.; 6 to 30° C.; 6 to 28° C.; 6 to 26° C.; 6 to 24° C.; 6 to 22° C.; 6 to 20° C.; 6 to 18° C.; 6 to 16° C.; 6 to 14° C.; 6 to 12° C.; 6 to 10° C.; 6 to 8° C.; 8 to 40° C.; 8 to 38° C.; 8 to 36° C.; 8 to 34° C.; 8 to 32° C.; 8 to 30° C.; 8 to 28° C.; 8 to 26° C.; 8 to 24° C.; 8 to 22° C.; 8 to 20° C.; 8 to 18° C.; 8 to 16° C.; 8 to 14° C.; 8 to 12° C.; 8 to 10° C.; 10 to 40° C.; 10 to 38° C.; 10 to 36° C.; 10 to 34° C.; 10 to 32° C.; 10 to 30° C.; 10 to 28° C.; 10 to 26° C.; 10 to 24° C.; 10 to 22° C.; 10 to 20° C.; 10 to 18° C.; 10 to 16° C.; 10 to 14° C.; 10 to 12° C.; 12 to 40° C.; 12 to 38° C.; 12 to 36° C.; 12 to 34° C.; 12 to 32° C.; 12 to 30° C.; 12 to 28° C.; 12 to 26° C.; 12 to 24° C.; 12 to 22° C.; 12 to 20° C.; 12 to 18° C.; 12 to 16° C.; 12 to 14° C.; 14 to 40° C.; 14 to 38° C.; 14 to 36° C.; 14 to 34° C.; 14 to 32° C.; 14 to 30° C.; 14 to 28° C.; 14 to 26° C.; 14 to 24° C.; 14 to 22° C.; 14 to 20° C.; 14 to 18° C.; 14 to 16° C.; 16 to 40° C.; 16 to 38° C.; 16 to 36° C.; 16 to 34° C.; 16 to 32° C.; 16 to 30° C.; 16 to 28° C.; 16 to 26° C.; 16 to 24° C.; 16 to 22° C.; 16 to 20° C.; 16 to 18° C.; 18 to 40° C.; 18 to 38° C.; 18 to 36° C.; 18 to 34° C.; 18 to 32° C.; 18 to 30° C.; 18 to 28° C.; 18 to 26° C.; 18 to 24° C.; 18 to 22° C.; 18 to 20° C.; 20 to 40° C.; 20 to 38° C.; 20 to 36° C.; 20 to 34° C.; 20 to 32° C.; 20 to 30° C.; 20 to 28° C.; 20 to 26° C.; 20 to 24° C.; 20 to 22° C.; 22 to 40° C.; 22 to 38° C.; 22 to 36° C.; 22 to 34° C.; 22 to 32° C.; 22 to 30° C.; 22 to 28° C.; 22 to 26° C.; 22 to 24° C.; 24 to 40° C.; 24 to 38° C.; 24 to 36° C.; 24 to 34° C.; 24 to 32° C.; 24 to 30° C.; 24 to 28° C.; 24 to 26° C.; 26 to 40° C.; 26 to 38° C.; 26 to 36° C.; 26 to 34° C.; 26 to 32° C.; 26 to 30° C.; 26 to 28° C.; 28 to 40° C.; 28 to 38° C.; 28 to 36° C.; 28 to 34° C.; 28 to 32° C.; 28 to 30° C.; 30 to 40° C.; 30 to 38° C.; 30 to 36° C.; 30 to 34° C.; 30 to 32° C.; 32 to 40° C.; 32 to 38° C.; 32 to 36° C.; 32 to 34° C.; 34 to 40° C.; 34 to 38° C.; 34 to 36° C.; 36 to 40° C.; 36 to 38° C.; and 38 to 40° C.

In one embodiment, the encapsulated islet preserved in a liquid for preservation or a liquid for transplantation is preferably subjected to transplantation as it is (without being transferred to another liquid). Omitting the operation of transferring from the liquid for preservation to another liquid for transplantation allows the encapsulated islet to be efficiently transplanted, or the time required for transplantation to be shortened. In addition, transplanting the encapsulated islet without transferring from the liquid for preservation to another liquid for transplantation can avoid the influence of the transfer on the encapsulated islet and maintain the function of the encapsulated islet to be transplanted in a higher state.

The subject to which the encapsulated islet is to be transplanted is arbitrary as long as it is a subject in need thereof. For example, the subject is a mammal, and specific examples thereof include a human, a dog, a cat, a horse, a cow, a pig, and the like, and preferably a human. In addition, the subject in need of transplantation of the encapsulated islet is preferably a subject suffering from diabetes, and is preferably a subject suffering from type I diabetes.

The transplantation site of the encapsulated islet is not particularly limited, and may be subcutaneous, intramuscular, omental, intraperitoneal, or the like, and is preferably transplanted intraperitoneally. The number of islet cells per administration is preferably about 5.0×10⁶ to 1.2×10⁸ cells/kg body weight, more preferably about 8.0×10⁶ to 8.0×10⁷ cells/kg body weight, and still more preferably about 1.2×10⁷ to 4.0×10⁷ cells/kg body weight per 1 kg body weight of the subject. In addition, the number of islets per administration is preferably about 5000 to 60,000 islets/kg body weight, more preferably about 8000 to 40,000 islets/kg body weight, and still more preferably about 12000 to 20,000 islets/kg body weight per 1 kg body weight of the subject.

### EXAMPLES

Hereinafter, the present invention will be described in more detail with reference to Examples, but the present invention is not limited thereto.

The usability of the encapsulated islet as a liquid for preservation or transplantation was evaluated in the following test.

### 1. Test Liquid

A liquid composition having the following table and the properties was used as a test liquid.

TABLE 1

| | |
|---|---|
| Na⁺ (mEq/L) | 130 |
| K⁺ (mEq/L) | 4 |
| Ca²⁺ (mEq/L) | 3 |
| Cl⁻ (mEq/L) | 109 |
| L-Lactate⁻ (mEq/L) | 28 |
| Trehalose (w/v %) | 3 |
| pH | 6.5 to 8.5 (approximately 7.4) |
| Properties (color | Clear and colorless liquid |
| tone and clarity) | |

### 2. Comparative Liquid for Preservation

To 500 mL of RPMI-1640 (manufactured by Gibco), 50 mL of inactivated Porcine Serum (manufactured by Gibco) and 5 mL of a stock solution of nicotinamide (1M nicotinamide) were added and mixed to prepare a comparative liquid for preservation.

### 3. Encapsulated Islet

The islet separated from the neonatal pig was suspended in a sodium alginate solution, and the alginic acid suspension solution was added dropwise to a 109 mM calcium chloride solution using a peristaltic pump to perform encapsulation. The capsules were collected, washed with saline, then 0.10% poly L-ornithine (PLO) solution was added, and mixed on a mix rotor for 10 minutes. The supernatant was removed, washing was performed with saline, and a 0.05% PLO solution was added and mixed on a mix rotor for 6 minutes. The supernatant was removed, washing was performed with saline, then a 10-fold diluted sodium alginate solution was added, and mixed on a mix rotor for 6 minutes. The supernatant was removed, washing was performed with saline, then 1.15% sodium citrate solution was added, and mixed on a mix rotor for 2 minutes. The supernatant was removed, washing was performed with saline, and then the supernatant was further removed to provide the encapsulated islet. The resulting encapsulated islet was suspended in a medium prepared by adding porcine serum, nicotinamide, and an antibiotic to RPMI-1640, and the suspension was dispensed into a T175 flask. Then, culturing was performed in a CO₂ incubator at 37° C. for 26 days or 30 days.

### 4. Test Animal

The B6 mouse diabetes model by STZ administration was used as a test animal. Mice having a blood glucose level at the time of satiation of 300 mg/dL or more on both second days and third days (day of transplantation) after STZ administration, and having no abnormality observed by visual observation of the general condition were subjected to stratification randomization allocation using a statistical analysis system EXSUS 10.0 (CAC EXICARE Corporation) on the body weight and the blood glucose level on the third day.

### 5. Test Configuration

The test was configured as shown in the following table.

TABLE 2

| | STZ dose | | Number of |
|---|---|---|---|
| Group name | (mg/kg) | Capsule dose | animals used n |
| Non-preserved | 200 | 8,000 (IEQ/body) | 8 |
| Control | 200 | 8,000 (IEQ/body) | 8 |
| Test | 200 | 8,000 (IEQ/body) | 8 |

In the non-preserved group, STZ was administered to the test animal at a dose of 200 mg/kg, and the encapsulated neonatal pig islet cultured until the 30th day was suspended in saline and intraperitoneally transplanted with 8,000 IEQ (Islet EQquivalent (1 IEQ) indicates one islet with a diameter of 150 μm) without undergoing the step of preservation in the test liquid or the comparative liquid for preservation. The encapsulated neonatal pig islet was prepared by the following procedure. Based on the IEQ count results on the day of transplantation, the culture flask was removed from the incubator immediately before transplantation, and each IEQ dose was dispensed into a 50 mL tube. This was allowed to stand at room temperature until the washing operation. The medium in the tube was removed with a pipette, and about 10 mL of HBSS (Gibco) under roan temperature conditions was added and allowed to stand, then the supernatant was removed. After repeating these operations 3 times, about 10 mL of saline under roan temperature conditions was added, suspended, and allowed to stand, then the supernatant was removed to about 4 mL, and transplantation was quickly performed.

In the control group, the encapsulated neonatal pig islet cultured until the 26th day was preserved (EMS-702G manufactured by FUKUSHIMA GALILEI CO. LTD.) at 4° C. for 96 hours in the comparative liquid for preservation described in the above “2. Comparative liquid for preservation”, then suspended in saline, and intraperitoneally transplanted with 8,000 IEQ. The encapsulated neonatal pig islet was prepared by the following procedure. Based on the IEQ count results at the start of preservation, the preservation container was removed from the refrigerator immediately before transplantation, and each IEQ dose was dispensed into a 50 mL tube. This was allowed to stand with cooling ice until the washing operation. The liquid for preservation in the tube was removed with a pipette, and about 10 mL of HBSS (Gibco) under room temperature conditions was added and allowed to stand, then the supernatant was removed. After repeating these operations 3 times, about 10 mL of saline under roan temperature conditions was added, suspended, and allowed to stand, then the supernatant was removed to about 4 mL, and transplantation was quickly performed.

In the test group, the encapsulated neonatal pig islet cultured until the 26th day was preserved (EMS-702G manufactured by FUKUSHIMA GALILEI CO. LTD.) at 4° C. for 96 hours in the test liquid described in “1. Test liquid” above, and then directly intraperitoneally transplanted with 8,000 IEQ. The encapsulated neonatal pig islet was prepared by the following procedure. Based on the IEQ count results at the start of preservation, the preservation container was removed from the refrigerator immediately before transplantation, and each IEQ dose was dispensed into a 50 mL tube. This was allowed to stand with cooling ice until the washing operation. The liquid for preservation in the tube was removed to about 4 mL with a pipette and transplantation was quickly performed.

Transplantation of the encapsulated islet was performed in the following procedure in all groups. Mice were introduced and maintained under anesthesia with an isoflurane vaporizer and the abdomen was disinfected with 70% ethanol spray. A predetermined dose of the encapsulated islet liquid was administered intraperitoneally via 16G surflo cannula. The skin of the puncture site was closed with a surgical adhesive (Aron Alpha A “Sankyo”, manufactured by DAIICHI SANKYO COMPANY, LIMITED). The body temperature decreased after the surgery, and thus heating was performed with a heater until awakening. The day of transplantation was represented as Day 0.

### 6. Observation, Measurement, Inspection

### 6-1. Observation of General Conditions

The general conditions including the confirmation of life and death were observed every day.

### 6-2. Body Weight Measurement

Body weight was measured on the day of STZ administration, the day of transplantation, twice a week thereafter, and the day of an oral glucose tolerance test (OGTT).

### 6-3. Measurement of Blood Glucose Level

On the day of STZ administration (immediately before administration), on the second day of administration, on the day of transplantation and thereafter, the mice were retained with a retention device twice a week, and blood glucose levels were measured by tail vein puncture blood collection (several μL) with a 27G injection needle. A niprostat strip (NIPRO) was used for the measurement. Blood glucose levels were measured in the morning.

### 6-4. Oral Glucose Tolerance Test (OGGT)

The test was performed after the measurement date of the blood glucose level at the time of satiation at the 4th week after transplantation. A 10% glucose aqueous liquid (Otsuka Pharmaceutical Factory, Inc.) was orally administered to a mouse fasted for about 16 hours from the previous day under the condition of 10 μL/g (1 g/kg of glucose) by sonde, and the transition of the blood glucose levels was evaluated. The blood glucose levels were measured before administration and for 0, 15, 30, 45, 60, 75, 90, 105, and 120 minutes after administration.

### 6-5. Necropsy

After completion of the OGTT, the abdomen was opened under isoflurane anesthesia, and an abnormality observed in the macroscopic findings was recorded. Blood was collected from the posterior vena cava using a syringe with a 26G needle. The blood was placed in a microtina blood collection tube (BD, EDTA-2K), mixed by mild inversion, and then preserved under ice cooling. The blood plasma was separated by centrifugation at 4° C. and 3000×g for 10 minutes, placed in a separate microtube, and preserved at −30° C. or less as a sample for porcine C-peptide measurement.

### 6-6. DTZ Staining of Collected Encapsulated Islet

A DTZ solution was added to the collected encapsulated islet suspension, and DTZ staining was observed with an inverted microscope (CKX41 manufactured by Olympus Corporation) and was photographed. The DTZ solution was prepared by adding 5 mL of DMSO (Sigma-Aldrich) and 45 mL of HBSS to 100 mg of Dithizone (FUJIFILM Wako Pure Chemical Corporation), sufficiently dissolving the mixture, and then sterilizing the solution with a 0.22 μm filter. Regarding spheroids in the visual field of the obtained images, spheroids with and without a portion stained with dithizone were counted, and the purity was calculated by the following calculation formula: “Purity (%)=(number of spheroids with a part stained with dithizone)/(total number of spheroids)×100”.

### 6-7. Measurement of Porcine C-Peptide

The preserved plasma was thawed at room temperature and the porcine C-peptide concentration was measured using the Mercodia Porcine C-peptide ELISA kit (Mercodia, product number: 10-1256-01).

### 6-8. SGS Assay

SGS assays were performed on the collected capsules. The amount of insulin secreted when allowing to stand for 1 hour in RPMI1640 containing 500 mg/L glucose (low glucose) or 5000 mg/L glucose (high glucose) was determined using Mercodia Porcine Insulin ELISA. Measurement was performed according to the kit package insert, and 1 mg=115 U was used for unit calculation. According to the SGS assay, SI (stimulation index), which is the ratio between the insulin secretion amount of low glucose and the insulin secretion amount of high glucose, was determined.

### 6-9. Statistical Processing

The average and standard deviation of the obtained data were determined and graphed as necessary. For statistical analysis, Student's t-test was performed using EXSUS 10.0. It was determined that p<0.05 indicated a statistically significant difference, and p<0.1 indicated a tendency toward significance.

### 7. Results

### 7-1. General Conditions

After encapsulated islet transplantation, animal number 15 (non-preserved group) and animal number 38 (test group) were euthanized because of behavioral inhibition. None of the other individuals satisfied behavior inhibition due to diabetes and a body weight loss of 20% or more as compared to at the time of transplantation, and no individual was subjected to euthanasia treatment.

### 7-2. Body Weight

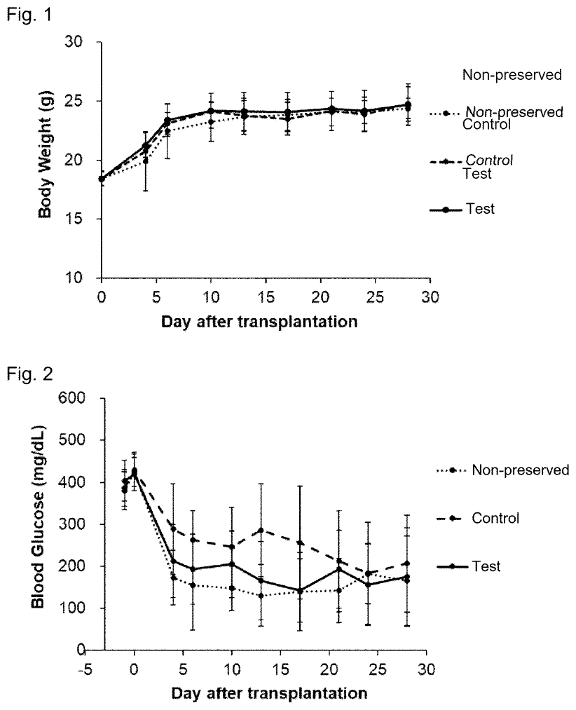

Body weight increased from capsule transplantation to the day of necropsy. No significant differences in average body weight were observed between the groups (FIG. 1).

### 7-3. Blood Glucose Level at the Time of Satiation

Blood glucose levels at the time of satiation decreased after capsule transplantation in all groups. After the 13th day from transplantation, the blood glucose level of the control group once increased, but decreased again, and finally, there was no significant difference between the groups (FIG. 2).

### 7-4. OGTT

Some individuals with poor glucose responsiveness were observed, but there was no large difference in all groups, and glucose responsiveness was shown.

### 7-5. Necropsy

At necropsy 1 month after transplantation, 1/7 cases (14.3%) in the non-preserved group, 2/8 cases (25.0%) in the control group, and adhesion of the capsule to the liver or capsule mass in the abdominal cavity were observed. In the test group, adhesion of the capsule to the liver or capsule mass in the abdominal cavity was not observed.

### 7-6. DTZ Staining of Collected Encapsulated Islet (Islet Purity)

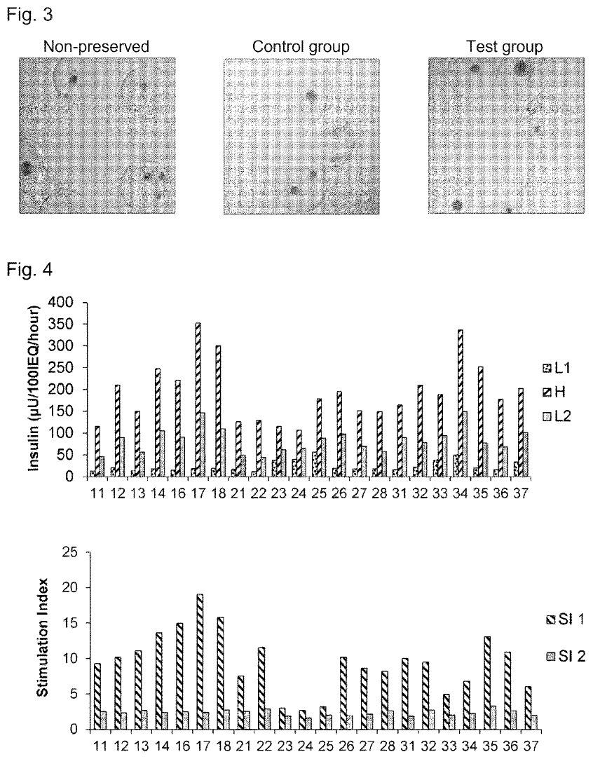

The encapsulated islet collected one month after transplantation confirmed DTZ-positive cells in all groups (FIG. 3). The positive rate was 96.95 in the non-preserved group, 91.5% in the control group, and 95.1% in the test group, and there was no difference between the groups.

### 7-7. Porcine Derived C-Peptide

Porcine derived C-peptide in mouse plasma was observed in all the individuals 1 month after transplantation. The average value in each group was 165.44 pmol/L in the non-preserved group, 178.72 pmol/L in the control group, and 174.75 pmol/L in the test group, and there was no difference between the groups.

### 7-8. Insulin Secretion and Glucose Responsiveness of Collected Encapsulated Islet (SGS Assay)

The encapsulated islet collected 1 month after transplantation in all individuals showed glucose responsiveness (FIG. 4). In FIG. 4, “L1” and “L2” indicate the amount of insulin secreted in allowing to stand in low glucose (500 mg/L), “H” indicates the amount of insulin secreted in allowing to stand in high glucose (5000 mg/L), “SI1” indicates the stimulation index of “H/L1”, and “SI2” indicates the stimulation index of “H/L2”. In addition, in FIG. 4, “11 to 18” represent the non-preserved group, “21 to 28” represent the subject group, and “31 to 37” represent the test group.

From the above results, it was shown that the test liquid is not only useful as a liquid for preservation for the encapsulated islet, but also adhesion of the encapsulated islet to tissue after transplantation can be suppressed by using the test liquid as the liquid for transplantation. The fact that the sane composition can be used as the liquid for transplantation and the liquid for preservation allows to omit the operation of transferring the preserved encapsulated islet to the liquid for transplantation before transplantation, and not only to improve the work efficiency but also to suppress the occurrence of contamination during liquid replacement, and thus handleability of the encapsulated islet is dramatically in-proved.

1. A liquid for preservation or transplantation of an encapsulated islet, comprising trehalose or a derivative thereof or a salt thereof.

2. The liquid according to claim 1, wherein the liquid is an isotonic liquid.

3. The liquid according to claim 2, wherein the isotonic liquid is Ringer's lactate liquid.

4. The liquid according to claim 1, wherein a concentration of trehalose or a derivative thereof or a salt thereof is 2.0 to 6.0 (w/v) %.

5. The liquid according to claim 1, being used for preventing tissue adhesion of the encapsulated islet.

6. A method for preservation of an encapsulated islet, comprising a step of preserving the encapsulated islet in the liquid according to claim 1.
